(19) United States
(12) Patent Application Publication
(10) Pub. No.: US 2016/0135765 A1
(43) Pub. Date: May 19, 2016

(54) ACTIVE WATER PHANTOM FOR THREE-DIMENSIONAL ION BEAM THERAPY QUALITY ASSURANCE

(71) Applicants: Steven E. Vigdor, Bloomington, IN (US); Minfang Yeh, Stony Brook, NY (US); David E. Jaffe, Riverhead, NY (US)

(72) Inventors: Steven E. Vigdor, Bloomington, IN (US); Minfang Yeh, Stony Brook, NY (US); David E. Jaffe, Riverhead, NY (US)

(73) Assignees: Phenix Medical LLC, San Francisco, CA (US); Brookhaven Science Associates, LLC, Upton, NY (US)

(21) Appl. No.: 14/547,334

(22) Filed: Nov. 19, 2014

(51) Int. Cl. A61B 6/00; G01T 1/20

(52) U.S. Cl. CPC A61B 6/4258; A61B 6/583; G01T 1/20

(57) ABSTRACT

An Active Water Phantom is designed to provide fast, accurate, high resolution, complete Quality Assurance of patient-specific treatment plans utilizing intensity-modulated Ion Beam Therapy, prior to their delivery to the patient. The detection medium is a tissue-equivalent water-based liquid scintillator material. A three-dimensional pattern of scintillation light, emitted upon ion beam irradiation, is reconstructed from three orthogonal two-dimensional light yield profiles, which are read out for each individual beam energy layer. The 3-D information has dose measurement accuracy 1-2% and spatial resolution 1-2 millimeters. The measurement sequence provides up to four orders of magnitude more data characterizing the treatment plan than currently commercially available alternatives, all in a time period no greater than that needed for actual delivery of the dose fraction to a patient. The system provides sophisticated control and readout of the cameras or photo-detectors, data archiving and analysis, simulation capabilities, and 3-D dose image reconstruction and visualization.

### STATEMENT OF GOVERNMENT RIGHTS

This invention was made with Government support under contract number DE-AC02-98CH10886, awarded by the U.S. Department of Energy. The Government has certain rights in the invention.

### FIELD OF THE INVENTION

The present invention is in the technical field of dosimetry for radiotherapy treatments for cancer. More particularly, the present invention is in the technical field of dosimetry for quality assurance of ion beam therapy treatment plans, based on the scanning of intense accelerator beams of small cross-sectional area (so-called “pencil” beams), and of adjustable energy and intensity, to irradiate the full volume of an arbitrary-shaped target tumor conformally, while providing minimal dose to surrounding healthy tissue.

### BACKGROUND OF THE INVENTION

In comparison to standard X-ray therapy, proton or heavier-ion beam therapy is capable of significantly improving dose localization by increasing dose delivered to the target volume while minimizing dose delivered to the surrounding tissue. These improvements are based on the finite penetration range of therapeutic ion beams in the target material. Furthermore, the energy deposition to the target material increases as the ion beam slows down and reaches a sharp maximum near the end of the penetration range. As a result, ion beam therapy has the potential to provide the best possible treatment option for control and elimination of tumors, with fewer short- and long-term toxic side effects.

The majority of ion beam therapy treatments to date have been delivered using legacy passive scattering systems, wherein the treatment dose field is formed through patient-specific apertures and range compensators. However, the inherent advantages of ion beam therapy are best exploited by an alternative approach, applying Pencil Beam Scanning (PBS) methods of dose delivery to achieve full 3-D conformity to any tumor volume without using apertures and compensators. Pencil Beam Scanning refers to a method where a small diameter incident ion beam is spread laterally across the tumor at a certain depth using scan magnets that sweep the beam in two lateral dimensions. The scan magnets are situated near the exit of a beam delivery gantry that can be rotated to irradiate the tumor from multiple directions. The beam intensity is varied for each 3-D spot (voxel) to achieve a dose distribution that conforms exactly to the tumor area at that depth. Repeating this process for a range of decreasing energies (energy stacking), adjusting the size of the 2-D scan to match the tumor area for each depth layer, allows treatment of the full tumor volume with any arbitrary shape. The beam intensity is varied for each 3-D spot (voxel) to achieve a dose distribution that conforms exactly to the tumor volume. The passive scattering and PBS approaches are contrasted schematically in FIGS. 1 and 2 and in realization of treatment plans for a given tumor in FIG. 3.

The PBS approach not only reduces the time and cost of individual patient treatments with ion beam therapy, but furthermore, as illustrated in FIG. 3, improves treatment precision and the range of tumors to which ion beam therapy is well suited. In combination with ongoing advances in accelerator, beamline and gantry design, adoption of PBS techniques allows ion beam therapy to compete favorably, both financially and clinically, with Intensity Modulated Radiation Therapy (IMRT) carried out with X-rays. However, PBS relies heavily on a high degree of accuracy in treatment planning and in the functioning of scanning magnets, complex control systems and software to implement the treatment plan. Consequently, clinical application of PBS demands sophisticated quality assurance (QA) dosimetry devices capable of verifying the three-dimensional dose distribution of each patient-specific treatment plan prior to its delivery to a patient.

The market demand is for a QA device capable of producing a three-dimensional image of the full treatment dose in a phantom that simulates a human body section, with spatial resolution on the order of 1-2 mm and dose profile information of order 1-2% relative precision, all in no more time than will be required for the patient's daily exposure to beam. If at all possible, the QA detector should provide such an image for every layer in the beam energy stacking individually as well as for the sum over all layers, in order to test the design and implementation of the PBS treatment plan completely.

Embodiments of the present invention are aimed at meeting this market demand with a device that would improve the resolution, accuracy, completeness and cost-effectiveness of the verification process in comparison with currently available technology. In particular, present QA technology typically requires physical movement of a gas ionization chamber array during several repeated dose measurements, and is limited by time constraints to measurements for only several energy layers. The time required for QA measurements with present technology represents a bottleneck that limits patient throughput at ion beam therapy clinics.

### BRIEF SUMMARY OF THE INVENTION

Embodiments of the present invention provide an Active Water Phantom that simulates an “ideal” patient and scintillates to reveal, in three dimensions, the radiation dose delivered by an ion beam to which it is exposed. This Active Water Phantom combines the traditional use by radiotherapy clinics of water as a preferred passive phantom, matching well the density and chemical composition of normal human body tissue, with the recent breakthrough development for particle physics purposes of a water-based liquid scintillator material (WbLS). (See for example, M. Yeh, et al., Nuclear Instruments and Methods A660, 51 (2011)). By using light generated in the WbLS itself by ion beam energy deposition, embodiments of the present invention eliminate the “middle man” detector, such as that used in the only system currently being marketed commercially for PBS quality assurance: the IBA DigiPhant system, in which a pixelized gas ionization chamber (with 7.6×7.6 millimeter pixels) is moved in discrete, time-consuming steps through a passive water phantom to provide two-dimensional lateral dose profiles, each with marginal spatial resolution, at several discrete depths within the water.

In a particular embodiment of the invention, the WbLS volume, contained in a water tank with three transparent (in addition to other opaque) walls, will be viewed by charge-coupled device (CCD) cameras, CID cameras, photo-detectors, photomultiplier tubes (PMT), avalanche photodiodes, or other photo-sensitive devices, from three orthogonal sides to provide three simultaneous two-dimensional projections of the scintillation light generated by the energy deposition of the ion beam stopping in the scintillator. At the dose rates typical of ion beam therapy treatments, sufficient statistical precision on these light projection profiles can be obtained in exposure times of the CCDs or other photo-sensitive devices that last for no more than 50-100 milliseconds. Furthermore, modern CCDs providing sufficient spatial resolution can be read out every 50 milliseconds, or even more frequently.

Depending on the details of the PBS implementation, a full lateral 2-D scan at one beam energy typically takes approximately 100-1000 milliseconds to complete, and two or more “repaints” of the target area may be needed to deliver the full dose at the corresponding depth layer. Consequently, the Active Water Phantom is capable of providing the three orthogonal profiles for every individual energy layer in the PBS treatment, and in some cases, even for individual scan lines within each 2-D layer. When combined with the known general shape of the ion beam energy deposition as a function of depth for each beam energy incident on the device, the information from the Active Water Phantom will provide an unambiguous full 3-D image of the delivered dose, based on measurements that take no more time than the actual (subsequent) dose delivery to the patient.

Embodiments of the invention also include a sophisticated software system executed by the control computer 185, which is configured to control the photo-sensitive readout devices, to archive and analyze their digitized electronic output signals and reconstruct from them the 3-D profile of light yield within the WbLS, and to reconstruct the radiation dose profile from the light yield profile. The reconstruction steps require careful inclusion of the measured quenching of WbLS scintillation light generated per deposited energy unit when the rate of energy deposition is maximized near the end of the ion beam's range, and of measured light scattering within the WbLS (anticipated to have little effect) and of the geometry of the device's optical system. The control computer 185 and associated software will also allow visualization of the determined 3-D dose profile for each energy layer individually, building up to the cumulative 3-D dose image.

In particular embodiments, the control computer 185 and associated software will also interface to PBS patient treatment planning software, to predict the light yield distribution that should be observed, and to allow rapid quantitative comparison of the predicted and measured light profiles for every energy layer in the treatment. On the basis of deviations between the measured and predicted 3D profiles, the software will provide radiation technologists with a simple go/no-go output signal regarding the adequacy of the treatment plan and implementation to meet clinical requirements. The criteria for clinical acceptability, incorporating accuracy of the targeting, absolute delivered dose and dose conformity to tumor shape, will be established by medical physicists working with PBS systems, taking into account the performance characteristics of the QA detector.

PCT Patent Pub. No. WO 2000/079302, entitled “Three-Dimensional Liquid Scintillation Dosimetry System”, by Kirov, discloses a dosimetry system with a liquid scintillation solution. U.S. Patent Pub. No. 2012/0168630, entitled “Liquid Scintillator For 3D Dosimetry For Radiotherapy Modalities”, by Beddar et al., discloses a liquid scintillator for a three-dimensional dosimetric measurement of a radiation beam. These two patent publications are incorporated herein by reference in their entireties. However, embodiments of the present invention differ in several ways that represent technological breakthroughs that provide a number of advantages over the dosimetry systems described in the above two patent publications. First among these breakthroughs is the usage of a water-based liquid scintillator, which provides much improved tissue equivalence (hence, dose accuracy), improved light attenuation length (hence, better spatial resolution), scintillation yield adjustability, and ease of handling, without the chemical, corrosiveness and combustibility hazards of conventional liquid scintillators.

The second breakthrough is the full consideration of phantom volumes large enough to contain the largest and deepest tumors that are treated clinically with ion beam therapy, including scintillator volumes more than two orders of magnitude greater than those considered in the above-cited Beddar et al. reference, for example. The larger volumes of clinical interest necessitate the more careful design described herein of an optical system that can provide the needed 1-2 mm spatial resolution over the full phantom volume, including the full necessary camera depth of field, without modification or movement of the detector during the QA measurement sequence. The care in the optical system must also be matched by precision in the dose image reconstruction software, in order to unambiguously unravel dose profile from light collection efficiency variations over the larger irradiation volume.

In yet another aspect, embodiments of the invention provide an Active Water Phantom that includes a water-tight tank with a plurality of transparent walls and a plurality of opaque walls. The Active Water Phantom also has a water-based liquid scintillator completely filling the tank. The water-based liquid scintillator is configured to simulate human tissue, and is also configured to emit scintillation light upon irradiation by an ion beam, to reveal the three-dimensional spatial dose distribution that would be delivered to a patient by the ion beam. A plurality of light-detecting devices provides three mutually orthogonal real-time measurements of the scintillation light intensity emitted from the water-based liquid scintillator. Each measurement is a function of a two-dimensional position. The Active Water Phantom includes processing electronics configured to digitize the signal outputs from each of the plurality of light-detecting devices.

In a particular embodiment, the plurality of light-detecting devices comprises one of a plurality of CCD cameras, CID cameras, avalanche photodiodes, and photomultiplier tubes. In a further embodiment, the Active Water Phantom includes a positioning device configured to adjust the location and orientation of the Active Water Phantom in concert with a position of an ion beam delivery gantry. The water-based liquid scintillator may be sized to accommodate the largest and deepest tumor locations to be treated by ion beam therapy.

In certain embodiments, the water-based liquid scintillator comprises a concentration of organic scintillating molecules attached to water molecules by means of a non-ionic surfactant bridge. In more particular embodiments, the organic scintillating molecules are linear alkylbenzene (LAB). Alternative embodiments may utilize other organic scintillating molecules, such as trimethyl benzene (pseudocumene or PC), di-isopropylnaphthalene (DIN), phenylxylylethane (PXE), or phenylcyclohexane (PCH). The water-based liquid scintillator may further comprise fluorescent material and/or other wavelength-shifting materials such that light emitted by the organic scintillating molecules is shifted to improve a match with the spectral sensitivity of the plurality of light-detecting devices. In an embodiment of the invention, the addition of the organic scintillating molecules, the fluorescent material and other wavelength-shifting materials produce a water-based liquid scintillator whose density differs from that of pure water by less than 1%. Furthermore, the water-based liquid scintillator material matches the composition percentages, by weight, of hydrogen, oxygen and carbon in normal human body tissue sufficiently well that ion beam energy deposition in the water-based liquid scintillator and in relevant human tissue can be matched to within 1%. The non-ionic surfactant bridge may be sulfonic acid. Further, the non-ionic surfactant may be optimized to chemically suppress radiation-induced free radical formation in the water-based liquid scintillator.

In some embodiments, the water-based liquid scintillator is configured such that less than 1% of the emitted light is attenuated by passage through the water-based liquid scintillator on its way toward each of the plurality of light-detecting devices. In a particular embodiment, the reflectance of an inner surface of each of the plurality of transparent walls and each of the plurality of opaque walls for wavelengths within the emission spectrum of said water-based liquid scintillator is less than four percent. In other embodiments, each of the plurality of transparent walls of said water tank transmits at least 80% of the scintillation light to its respective one of the plurality of light-detecting devices, while each of the plurality of opaque walls prevents external light from entering an interior volume of said water tank. Each of the plurality of transparent walls may be configured to act as optical lenses to transport light, generated at different distances from the transparent walls, along a line perpendicular to the transparent wall, with a depth of field as deep as 30 centimeters, to illuminate on its respective remote camera sensor a single spot whose dimensions are comparable to, or smaller than, the sensor pixel size, or pixel bin size, used.

The Active Water Phantom may include a plurality of optical fibers optically coupled to one or more of the plurality of transparent windows to transmit the generated scintillation light to remote photo-detectors. In certain embodiments, scintillation light is transported from the water tank to the plurality of light-detecting devices through hoods configured to eliminate light from sources external to the water tank, the hoods having inner wall surfaces designed to minimize reflections of the generated scintillation light.

In certain embodiments, each of the plurality of light-detecting devices is configured with a sufficient number of channels, and the optical transport system has sufficient resolution, to provide a two-dimensional view of the dose field that distinguishes light originating from neighboring pixels separated by no more than 1-2 millimeters within the water-based liquid scintillator, when the light origination points, within those pixels, occur at distances from the plurality of light-detecting devices varying over a depth of field as large as 30 centimeters.

The processing electronics may be configured to digitize output signals from each of the plurality of light-detecting devices in a sufficient number of bits, and with sufficiently low readout noise, to span a maximum-to-minimum dynamic range of approximately 1000:1 in the light yield generated from a given pixel. In some embodiments, the digitized output signal from each channel of the plurality of light-detecting devices is proportional within approximately 1% to the light yield from the corresponding pixel within the water-based liquid scintillator, up to the maximum light yield anticipated for ion beam therapy treatment plans. The digitized output signals from all channels may be read out and stored at least ten times per second, to provide 3-D dose profile measurements for each independent energy layer in a Pencil Beam Scanning treatment plan. The exposures of the plurality of light-detecting devices may be triggered externally by signals related to the ion beam being incident on the phantom, or triggered internally according to a software-selected preset sequence of exposures.

In a particular embodiment, a control computer is configured to provide simultaneous exposures from the plurality of light-detecting devices, wherein exposure lengths are determined either by software input or by the width of external trigger signals, and wherein a control computer is configured to store data from the plurality of light-detecting devices at readout rates of at least 10 frames per second.

The concentration of scintillating molecules is arranged, and the properties of the optical system and of the plurality of light-detecting devices are adjusted, in order to provide a statistical precision of approximately 1% in the measurement of the maximum anticipated light yield from a pixel within the water-based liquid scintillator, in exposure times of 100 milliseconds or less.

A control computer may be configured to simulate, based on a given ion beam therapy treatment plan, each two-dimensional light yield profile projection as the light yield profiles would be collected by the plurality of light-detecting devices. In some embodiments, the simulated light yield profile projections correspond with those light yield profiles measured by each of the plurality of light-detecting devices within approximately 2% of the measured light yield profiles, when all of the hardware and software controlling the pencil beam scanning delivery are functioning correctly. More generally, the control computer may be configured to provide a quantitative comparison of the measured light yield profiles and simulated light yield profile projections. In certain embodiments, the control computer is configured to provide offline reconstruction and visualization of a three-dimensional image of the full treatment dose field that generated the three orthogonal two-dimensional light profile views provided for each energy layer of the treatment plan by the plurality of light-detecting devices and said processing electronics.

The control computer may also be configured to generate a verification signal, for a treatment plan implementation, that indicates whether or not the treatment plan meets clinical acceptance criteria, based on the detailed comparison of the 3-D dose profiles, for each energy layer individually and for the sum of all energy layers, reconstructed from the Active Water Phantom measurements with those anticipated in the treatment plan.

### BRIEF DESCRIPTION OF THE DRAWINGS

The accompanying drawings incorporated in and forming a part of the specification illustrate several aspects of the present invention and, together with the description, serve to explain the principles of the invention. In the drawings:

FIG. 1 is a schematic plan view of a conventional passive scattering system for delivery of an ion beam of fixed energy and intensity to irradiate a tumor with the aid of patient-specific apertures and compensators;

FIG. 2 is a schematic plan view of a pencil beam scanning system for delivery of an ion beam of variable energy and intensity to irradiate a tumor;

FIG. 3 is a schematic illustration showing a comparison of treatment plans for two different approaches to proton therapy dose delivery for a tumor of complex shape wrapped around a critical organ;

FIG. 4 is a graphical drawing showing experimental results illustrating the light emission spectrum and light attenuation curves for a water-based liquid scintillator such as that found in embodiments of the invention;

FIG. 5 is a graphical illustration showing experimental results for the relative light yield from the water-based liquid scintillator as a function of the liquid scintillator concentration in the material;

FIG. 6 is a graphical drawing of one embodiment of the Active Water Phantom dosimetry system, in accordance with an embodiment of the invention;

FIG. 7 is an exploded view showing the construction of a water-based liquid scintillator tank, constructed in accordance with an embodiment of the invention;

FIG. 8 is a graphical illustration showing geometric optical calculations illustrating the precision of image formation at the camera position, as a function of beam distance from the camera, for one particular design of aspheric lens windows for the water-based liquid scintillator tank; and

FIG. 9 is a graphical illustration showing geometric optical calculations illustrating the displacement parallel to the camera axis of images formed by one particular design of aspheric lens windows for the water-based liquid scintillator tank, as a function of the light source position within the tank.

While the invention will be described in connection with certain preferred embodiments, there is no intent to limit it to those embodiments. On the contrary, the intent is to cover all alternatives, modifications and equivalents as included within the spirit and scope of the invention as defined by the appended claims.

### DETAILED DESCRIPTION OF THE INVENTION

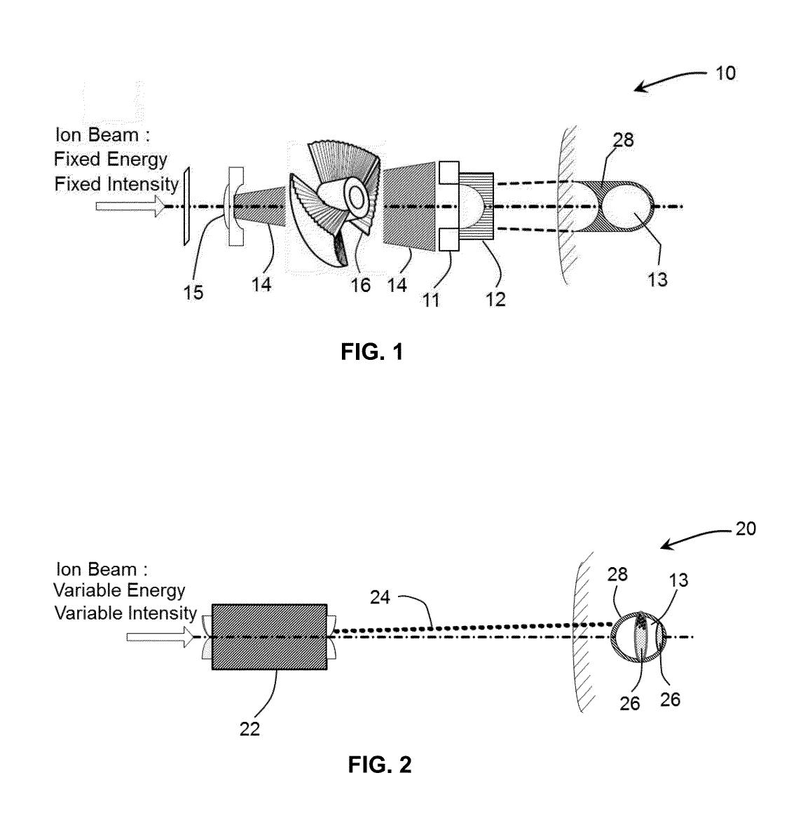

FIG. 1 shows a schematic layout of a conventional fixed-energy, fixed-intensity passive scattering system 10 for ion beam therapy delivery to a target volume, or tumor 13, using patient-specific apertures 11 and compensators 12 to form the desired radiation field from the broad beam 14 produced via scattering foils 15 and a range modulator 16. For comparison, FIG. 2 is a schematic view showing a system 20 for delivery of variable-energy, intensity-modulated ion beam therapy, using a scanning system 22 to scan a pencil beam 24 across depth layers 26 of a tumor. Two representative depth layers 26 are indicated in the figure. The shaded areas 28 in FIGS. 1 and 2, schematically indicating dose distributions outside the tumor volume, illustrate how pencil beam scanning can lead to reduced irradiation of healthy tissue adjacent to the tumor.

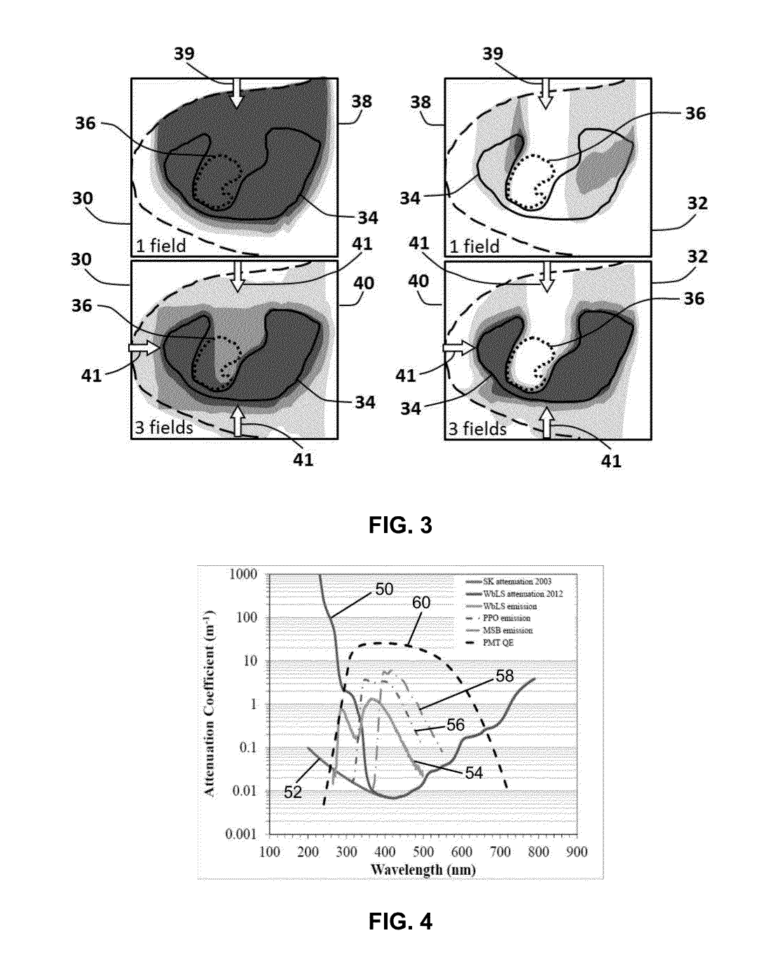

FIG. 3 is a schematic illustration which compares proton therapy dose delivery plans for the same two approaches shown schematically in FIGS. 1 and 2. Similar proton therapy dose delivery plans are discussed in An Overview of Compensated and Intensity-Modulated Proton Therapy”, A. J. Lomax, American Association of Physicists in Medicine (AAPM) Summer School (2003), the entire teachings and disclosure of which is incorporated herein by reference thereto. The left-hand frames 30 illustrate the dose strength that would be delivered by a passive scattering treatment while the right-hand frames 32 illustrate that for a pencil beam scanning modality, in both cases for the same tumor 34 of complex shape wrapping around a critical organ 36. In each case, the upper frames 38 show shaded dose intensity contours that can be attained with a single dose field (with beam incident in the direction indicated by the arrow 39), while the lower frames 40 show shaded dose contours attainable with three distinct dose fields, delivering beam in successive treatment stages along the directions indicated by the three arrows 41. The darkest shading of the contours in all four frames corresponds to a high delivered dose, and the lightest shading to a low delivered dose. Regions with no shading receive negligible doses. FIG. 3 clearly illustrates the promise of pencil beam scanning for sparing healthy critical organs adjacent to complex tumors from excessive radiation dose.

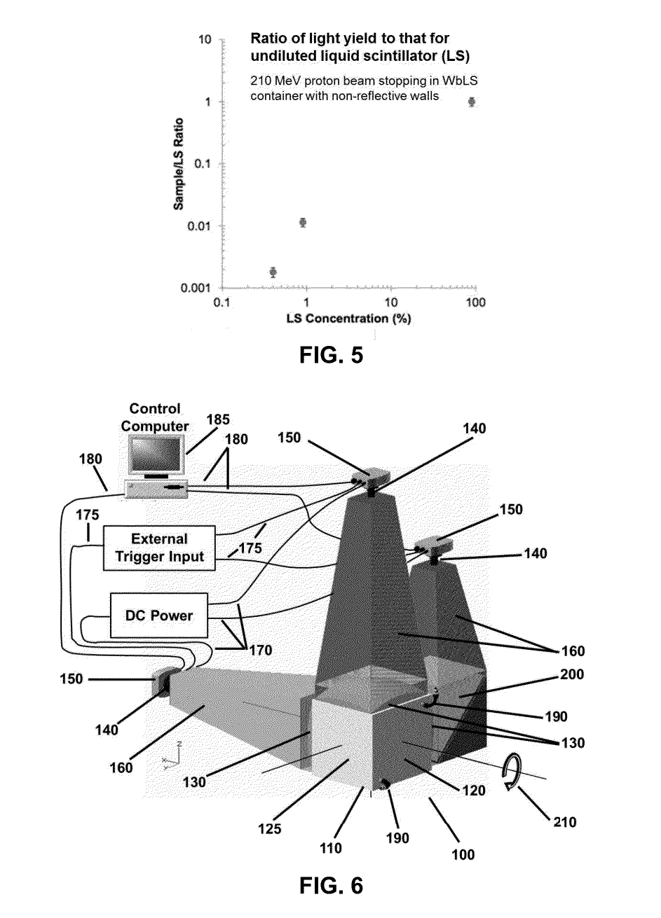

The graphs of FIGS. 4 and 5 show some relevant properties measured for one embodiment of the water-based liquid scintillator material developed by a Brookhaven National Laboratory team under the leadership of two of the present inventors (Yeh and Jaffe). In some embodiments, the liquid scintillator includes material that comprises a small concentration of organic scintillating molecules attached to water molecules by means of a non-ionic surfactant bridge, to produce a WbLS with long-term chemical stability. In the particular embodiment for which results are shown in FIGS. 4 and 5, the scintillating molecules are linear alkylbenzene (LAB) added to the water in varying concentrations and the surfactant is sulfonic acid. It should be understood that the present invention is intended to encompass embodiments utilizing WbLS based on alternative surfactants and organic scintillating molecules, including, but not limited to trimethyl benzene (pseudocumene or PC), di-isopropylnaphthalene (DIN), phenylxylylethane (PXE), and phenylcyclohexane (PCH). For a 1% LAB concentration, the curve 50 in FIG. 4 indicates the attenuation coefficient associated with light scattering and absorption in the WbLS, as a function of the light's wavelength. For wavelengths above about 380 nanometers, and throughout the visible light region, this curve overlaps the curve 52 representing results for a highly purified large water sample in the SuperKamiokande neutrino detector in Japan.

FIG. 4 also shows the curve 54 representing the spectrum of scintillation light emitted by the LAB molecules, and how that spectrum is subsequently shifted toward higher wavelengths by mixing into the water small amounts of a fluorescent material (curve 56) and of an additional wavelength-shifting material (curve 58). The final light output spectrum represented by curve 58 peaks near 400 nanometers, in the blue region of the visible light spectrum, with small contributions from the near ultraviolet. In the vicinity of this peak, the attenuation length shown in curve 50 exceeds fifty (50) meters, indicating that less than 1% of the produced light will be lost to scattering or absorption by passage through the maximum length (less than 50 centimeters) of WbLS material needed to simulate the largest and deepest tumor volumes for which ion beam therapy will be applied. This very low attenuation is a significant advantage of the WbLS, implying that light yield measurements as a function of source position will accurately reflect the profile of energy deposition that caused the scintillation.

The quantum efficiency for converting photons to electrons in the photo-sensitive devices used to collect the emitted light must overlap with the emission spectrum represented by curve 58 in FIG. 4. This is indeed the case, as indicated by curve 60 in FIG. 4, for the photomultiplier tube used by the Brookhaven National Laboratory group that developed the WbLS in beam tests of its light yield. Some results of those tests are shown in FIG. 5, which plots the measured light yield as a function of LAB concentration, and as a ratio to the light yield for 100% LAB concentration. The measurements were made by stopping a 210 mega-electron volt proton beam in WbLS housed in a container with non-reflective Teflon walls. The beam energy is within the range used for proton beam radiotherapy.

The results in FIG. 5 indicate that a 1% LAB concentration produces about 1% as much light as pure liquid scintillator, or about 90 photons per mega-electron volt of energy deposition. In usage as a QA device for proton beam therapy, the Active Water Phantom 100 (shown in FIG. 6) would be exposed typically to about 10¹⁰ incident protons/second, each depositing 100 or more mega-electron volts. In a 100 millisecond exposure, each camera 150 (shown in FIG. 6) would then collect a tiny fraction of the roughly 10¹³ photons produced. Adequate photostatistics to achieve the dose measurement precision goal for the Active Water Phantom 100 could then be readily obtained with approximately 1%-4% liquid scintillator concentrations.

At the anticipated liquid scintillator concentration levels, the properties of the WbLS remain very close to those of pure water, and hence of the normal human body tissue that the Active Water Phantom 100 (shown in FIG. 6) is intended to simulate. For example, the WbLS material represented in FIG. 4, including fluorescent and wavelength-shifting admixtures, has 99.5% of the density of pure water. Its chemical composition (65.9% hydrogen atoms, 30.9% oxygen atoms and 3.1% carbon atoms, with small amounts of sulfur, nitrogen and other elements) is sufficiently close to that of pure water and of a typical adult human body (atomic fractions of approximately 62% hydrogen, 24% oxygen, 12% carbon, 1% nitrogen) to provide an excellent material to simulate the energy deposition and the scattering of ion beams in human tissue. Only tiny corrections will be needed to translate measured ion beam ranges in the Active Water Phantom 100 into the water-equivalent depths used as the normal calibration standard in ion beam therapy.

One further property of the WbLS material that will be important for its application in the Active Water Phantom 100 is radiation tolerance. A typical radiation dose delivered to a patient in a fractionated ion beam therapy treatment is 2 Gray or 200 Rad. A PBS treatment room in a fully subscribed clinic is likely to treat about 300 patients per year. Hence, an Active Water Phantom 100 used for quality assurance on all of these treatment plans may be exposed to as much as 1000 Gray or 100,000 Rad per year. In order to avoid the need to flush and refill the detector too frequently, it is desirable that the WbLS light yield will not be altered by more than about 10% by such annual radiation exposures. It is anticipated that the most important contribution to possible deterioration in light output will arise from the radiation-induced formation of free radicals in the water and the subsequent recombination of those free radicals with other molecules. In order to achieve optimal suppression of such free radical effects, it may be important to mix further anti-oxidants into the WbLS, or perhaps to explore the choice of alternative types or amounts of surfactant material.

Referring now to the invention in more detail, in FIG. 6 there is shown an exemplary schematic embodiment of the Active Water Phantom 100, comprising a nearly cubical WbLS tank 110 that will be filled with the WbLS material, with three opaque walls 120 and 125 and three transparent lens-windows 130. Some of the scintillation light transmitted through the lens windows 130 is detected by a plurality of light-detecting devices 150. While the embodiments of the invention described below include CCD cameras, it should be understood that the present invention is intended to encompass embodiments having any suitable light-detecting device 150 including, but not limited to CID cameras, photomultiplier tubes, avalanche photodiodes, etc. In the embodiment of FIG. 6, light transmitted through the lens windows 130 is collected by camera lenses 140 mounted to CCD cameras 150 at the ends of opaque camera hoods 160 that shield the cameras 150 from light generated by external sources. Although the lens windows 130 are transparent and the camera hoods 160 are opaque, both are drawn as semi-transparent in FIG. 6 for visual clarity. In particular embodiments, the interior surfaces of the camera hoods 160 are designed to minimize reflections of the generated scintillation light.

Referring still to FIG. 6, each of the CCD cameras 150 has three types of input/output signal. Input signals 170 transport to the cameras 150 the DC voltage needed to operate them. Input signals 175 carry the externally generated trigger logic signals that tell them when to initiate and when to terminate exposures in synchronization with ion beam irradiation. These trigger signals may be generated by an independent computer used to control the pencil beam scanning procedure at a proton therapy clinic. The same signals will also be available as input to the Active Water Phantom's control computer 185, to ensure proper communication and synchronization between the beam control and phantom control operations. Camera input/output signals 180 communicate with the Active Water Phantom's control computer 185, carrying both the computer-generated control signals that govern the cameras' 150 functioning and the digitized output signals generated by the cameras 150 to measure the charge accumulated in each pixel by the total number of photons that were incident during the exposure time.

Referring still to the invention in more detail in FIG. 6, the ion beam will enter the Active Water Phantom 100 through a thin opaque wall 125 and will stop within the contained WbLS tank 110 volume. In order for the same device to work for all patients, the depth of the tank 110 along the beam direction must be sufficient to accommodate the deepest tumors to be treated with ion beam therapy. Typically, a depth of 35 centimeters of WbLS will suffice. The transverse dimensions of the tank 110 must accommodate the largest tumor areas to be treated, typically 25 centimeters in each lateral dimension. Tubing 190, that will connect the tank 110 to external supply sources and receptacles (not shown in the figure) for filling and emptying the WbLS tank 110, is attached to one of the other opaque tank walls 120.

Referring still to FIG. 6, two of the three orthogonal light field images are viewed directly by CCD cameras 150 mounted to one side of and above the tank 110. In the embodiment in FIG. 6, the third orthogonal light field image is viewed from above via reflection by a planar mirror 200 mounted directly behind the tank 110. The reason for this arrangement is that nuclear reactions induced by the ion beam stopping within the WbLS are anticipated to produce a significant flux of forward-going neutrons that would impinge on, and cause radiation damage to, a CCD camera 150 mounted behind the tank 110, along the ion beam direction. In order to minimize radiation damage to all three CCD cameras 150 that might be caused by stray neutrons produced by the ion beam upstream of the Active Water Phantom 100, they will also be enclosed in radiation shielding that is not shown in FIG. 6.

Referring still to the invention in more detail, the entire assembly of tank 110, camera hoods 160, cameras 150 and lenses 140 shown in FIG. 6 will be enclosed within a mechanical housing that is not shown in the figure. The cables 170, 175 and 180 will be patched to their destinations via connectors on the housing walls. In some embodiments, the housing will have the capability to allow rotation of the Active Water Phantom 100 about one axis through the center of the tank 110 at mid-depth, as indicated by the curved arrow 210 in FIG. 6, in order that the same front opaque tank wall 125 will be presented to the ion beam for different angle settings of the gantry delivering the ion beam to a patient. This feature allows quality assurance of the full treatment plan for any plan involving multiple dose fields, as in the lower right-hand frame of FIG. 3. In some embodiments, the rotational capability can be provided by attaching the Active Water Phantom housing directly to the gantry snout. In other embodiments, the Active Water Phantom 100 may have its own stand, which permits the indicated rotation 210.

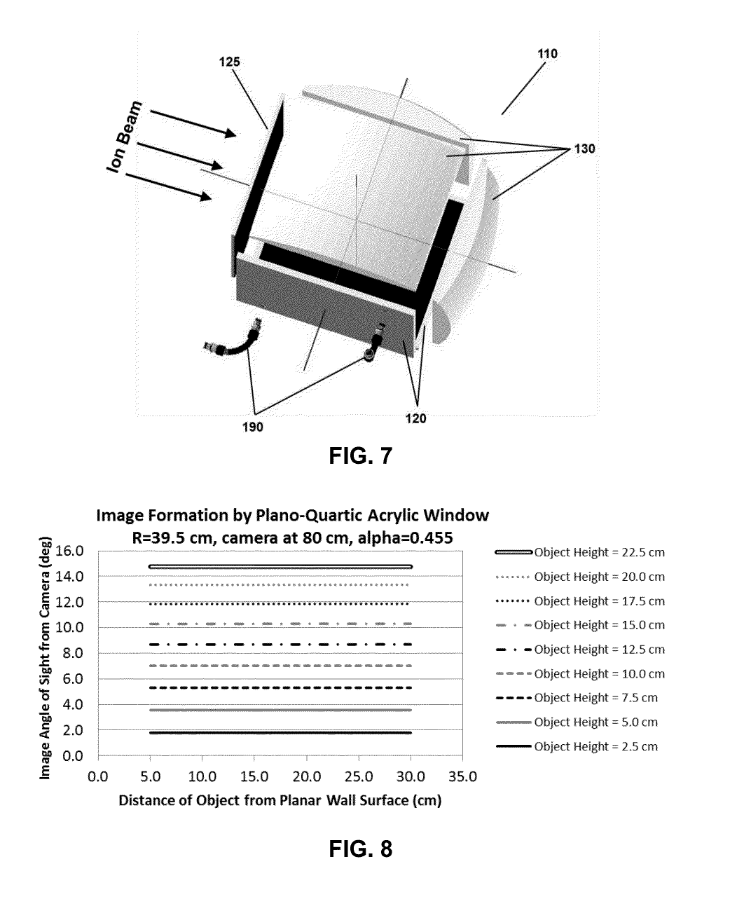

FIG. 7 shows an exploded view from above of the WbLS tank 110. All materials that come in contact with the WbLS must be robust against long-term deterioration via chemical interaction with or adsorption of the scintillating material (e.g., benzene). For the particular embodiment of WbLS material represented in FIG. 4, with LAB scintillant, it is already known that acrylic, some acrylic adhesives and polytetrafluoroethylene (PTFE, or Teflon by brand name) are acceptable materials. These are thus the primary tank wall materials used in the particular embodiment of FIG. 7. The walls of the tank 110 are all made primarily of acrylic, and the various walls are glued together with an acrylic cement, such as IPS Weld-On 4 compound. The opaque walls 120 and 125 are made from opaque acrylic sheet with thin black PTFE sheet glued to the inner faces with an acrylic adhesive such as 3M 300 LSE. This construction keeps external light out of the WbLS volume and minimizes reflections of internally generated light from the inner wall surfaces, in order to avoid confusion that could be caused by reflected images. The thickness of the beam entry wall 125 is designed to be no greater than about 6 millimeters to keep beam energy loss and scattering in the entry window to acceptable levels. The other opaque walls 120 may be made thicker to ensure mechanical robustness of the glued tank 110.

Still referring to FIG. 7, the filling and emptying tubes 190 can be made from bendable black acrylic tubing, and can be coupled to the WbLS tank 110 and to external valves and plumbing via compression fittings made especially for use with acrylic tubing. The tubes 190 can be valved off at an elevation above the top of the tank 110, for at least one of the tubes 190 in any orientation of the Active Water Phantom, to ensure complete filling of the tank 110 and to allow for an expansion volume to accommodate possible temperature changes of the WbLS.

Still referring to FIG. 7, the three transparent tank walls 130 (again drawn as semi-transparent for purposes of visual clarity) serve a special purpose needed to accommodate the significant depth of field (25-35 centimeters) over which each CCD camera 150 must record a well-focused image of the light field projection. When the Active Water Phantom 100 is in actual usage for quality assurance of ion beam therapy treatment plans, each energy layer in the treatment will involve scanning a beam of fixed energy in two dimensions across the entry tank wall 125. For a given beam energy, the beam will reach the end of its range at the same depth within the WbLS for each 2-D position within the scan, giving rise to a sharp-edged Bragg peak in the dose profile as a function of depth. Quality assurance of the tumor targeting is best provided if this sharp physical feature for each energy layer is preserved as a sharp optical feature of the light field images viewed by the side and top cameras in FIG. 6, even though the Bragg peak depth subtends different viewing angles at the camera location for different positions in the 2-D scan. Similarly, location of the sharp lateral edges of the dose profile for each energy layer via the rear light field projection, viewed through mirror 200, is an important aspect of the QA measurements, and it is desirable for a given lateral location to appear at a fixed lateral position in the camera's image, independent of the distance of the Bragg peak for that energy layer from the rear tank window.

In order to arrange for these desirable image features, the transparent tank windows 130 are designed to serve as lenses for the scintillation light, which create virtual images in which light originating in reality along a line in the WbLS parallel to the camera axis appears instead to originate along a fixed line-of-sight (i.e., at fixed viewing angle) to the distant camera. In this way, any point along the actual source line will show up within a given pixel (or bin of neighboring pixels) on the CCD camera, and sharp physical features will be manifested as sharp optical features.

In practice, the dimensions of the WbLS tank 110 are sufficiently large that the desired lens performance requires aspheric outer surfaces for the transparent acrylic windows 130, to minimize spherical aberration effects. In the particular embodiment shown in FIG. 7, the lens windows 130 are to be made from injection-molded UV-transparent acrylic with highly polished surfaces of a shape that optimizes image resolution in the distant cameras that view them. In one embodiment, the inner planar surfaces of the lens windows 130 can be covered with a thin magnesium fluoride coating of thickness designed to minimize light reflections from that surface for the peak wavelength of light emission from the WbLS. Magnesium fluoride is chosen as the anti-reflection coating material because its index of refraction is intermediate between those of the WbLS and the acrylic window material, but its material compatibility with the WbLS must be verified. In particular embodiments of the invention, the reflectance of the inner wall surfaces for each of the lens windows 130 or transparent walls, is less than four percent. Some of the transparent lens windows 130 may be made with a rectangular slab acrylic ledge on one or two edges to facilitate gluing to the other acrylic tank walls.

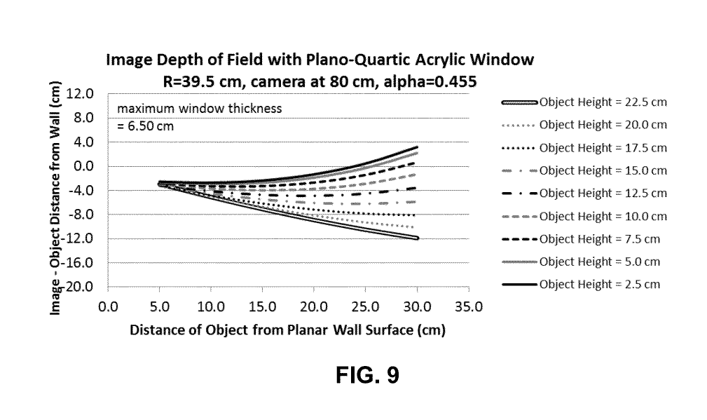

FIGS. 8 and 9 show the results of optical ray-tracing calculations of the image formation, as a function of the location of the light source within the WbLS, for one particular embodiment of the aspheric lens windows 130. In this embodiment, the window has a planar inner surface and a convex outer surface described by a quartic polynomial in the distance from the central lens axis. In a particular embodiment, the parameters of the polynomial are optimized to produce the best images over the full 25-centimeter depth of field and the full 35 centimeter by 25 centimeter field of view in side- and top-view cameras that are positioned 80 centimeters distant from the outermost point on the corresponding lens window surface. The index of refraction of the WbLS is assumed to be n=1.34, while that of the acrylic window material is n=1.49. FIGS. 8 and 9 illustrate the issues that need to be addressed in a careful design of the optical system for the Active Water Phantom 100.

FIG. 8 shows the angle of sight subtended at the center of the camera's lens 140 by light source points within the WbLS, as a function of the perpendicular distances of the source point from the planar window surface (on the horizontal axis) and from the central camera axis (designated as “object height” in the figure). By symmetry, any point along the central axis (i.e., at zero object height) would show up at zero degrees angle of sight. Each line in the graph of FIG. 8 corresponds to a different object height, covering the full relevant range of values. For a simple planar window, each of the lines in the figure would slant significantly downward toward smaller angles of sight as the distance of the source point from the planar window surface increases. The lens window 130 thus serves to make all source points at a given object height appear to lie along a common angle of sight to the camera. In fact, if one looks at the calculation results more microscopically, in the worst case in FIG. 8, the images at the near and far ends of the depth of field will appear to shift by about 9 microns on the CCD sensor, corresponding to an apparent shift of the source point within the WbLS of about 0.35 millimeters. This particular embodiment of the lens windows 130 is thus more than adequate to satisfy the spatial resolution (approximately 1-2 millimeters) demands of ion beam therapy QA.

The image formation by the tank's lens windows 130 also alters the apparent distance of the light source from the camera, and hence has implications for the depth of field that must be provided by the camera's own lens 140 to achieve the desired spatial resolution over the full target volume. This effect is illustrated in FIG. 9. For the same lens window design as considered in FIG. 8, FIG. 9 shows the apparent source displacement perpendicular to the planar surface of the lens window 130, again as a function of distance of the real source from the planar window surface and from the central camera axis. At the near end of the depth of field, the image formed by the lens window 130 always appears a few centimeters closer to the camera than the real source point. But at the far end of the depth of field, the image may appear anywhere from 4 centimeters further away to 12 centimeters closer to the camera, depending on the object height.

The net effect of the calculations in FIG. 9 is that, for source points near the central camera axis, the camera's lens must accommodate a somewhat larger depth of field: 31 centimeters rather than the 25 centimeter range corresponding to the largest lateral dimension of tumors to be treated by ion beam therapy. This depth of field can be easily realized with commercial camera lenses, but it influences the iris diameters that can be used with the camera, and therefore, the intensity of collected light. Furthermore, the position-dependent longitudinal image displacements represented in FIG. 9 imply small variations in light collection efficiency across the depth of field, which may necessitate small iterative software corrections to the reconstructed 3-D dose image.

Calculations of light collection efficiency indicate that a well-designed Active Water Phantom can readily achieve all of the desired performance parameters for ion beam therapy QA: photostatistics adequate to determine radiation doses with 1-2% precision for voxels with the highest dose; exposure times of 100 milliseconds or less to provide 3-D image reconstruction for each energy layer in a pencil beam scanning treatment; spatial resolution of 1-2 mm over the largest tumor volumes envisioned; dynamic range appropriate to measure maximum-to-minimum dose ratios of approximately 1000:1; QA of the full treatment plan in a time interval no longer than the actual patient treatment would require. This performance can furthermore be achieved with liquid scintillator concentration levels in the WbLS of approximately 1-4%, and with less than 1% loss of scintillation light to scattering and absorption within the WbLS.

In order to accommodate the signal-to-noise ratio needed to meet these performance characteristics, in particular the dynamic range requirement, it is desirable that the charge collected in each camera pixel (or bin of pixels if they can be grouped but still retain sufficient spatial resolution) be digitized in at least 12 bits, and that pixel readout noise be limited to no more than about 0.1% of the full-scale digitized signal. In addition, to ensure linearity of the digitized signal with the number of photons incident on the camera, each pixel or bin of pixels must accommodate a maximum charge (the so-called “pixel well depth”) comparable to or larger than the full-scale digitized value. In some embodiments, the digitized output signal from each channel of the plurality of light-detecting devices 150 is proportional within approximately 1% to the light yield from the corresponding pixel within the water-based liquid scintillator, up to a maximum light yield anticipated for ion beam therapy treatment plans. The digitized output signals from all channels may be read out and stored at least ten times per second, to provide 3-D dose profile measurements for each independent energy layer in a Pencil Beam Scanning treatment plan.

In order to achieve the desired spatial resolution, it is desirable to subdivide the irradiated WbLS volume into cubic voxels that are no larger than 1 millimeter on a side. For a maximum lateral dimension of 30 centimeters and depth of 35 centimeters for the phantom volume, each snapshot by each camera will then provide at least 100,000 channels of information, each containing 2 bytes of information on light intensity. Each snapshot then produces at least 600 kilobytes of data for the three cameras combined. A typical single dose field in an ion beam therapy treatment will involve about 20 seconds of beam exposure, or about 200 snapshots of 100 milliseconds duration apiece. Quality assurance measurements for each dose field with the Active Water Phantom will then produce more than 100 megabytes of data. In comparison, the current commercial alternative for pencil beam scanning QA—the IBA DigiPhant system—produces 2 bytes of information for each of the approximately 1000 channels of ionization chamber pixels, and for each of up to 10 discrete depths of the detector within a passive water phantom, or a total of about 20 kilobytes of data for a single dose field. The improvement by nearly four orders of magnitude in data quantity with the present invention promises also an enormous gain in the quality of the QA measurements, all to be obtained in shorter measurement times.

In alternative embodiments of the Active Water Phantom, it may be desirable for some applications to replace the CCD sensor readout with multi-channel photomultiplier tube (PMT) or avalanche photodiode (APD) readout. PMTs and APDs can be read out much faster than CCDs, allowing possible applications where one uses the Active Water Phantom information for real-time feedback to the beam scanning controls system. To achieve the desired voxel size in such an alternative embodiment, one would need bundles of optical fibers transporting scintillation light from the transparent windows of the WbLS tank 110 to the multi-channel PMTs or APDs. The transparent window design could then also change. It may be possible to achieve the desired spatial resolution in three dimensions with fewer than 100,000 PMT or APD channels by exploiting the division of collected light intensity among neighboring channels to improve effective pixel size.

In another alternative embodiment, the CCDs could be replaced by charge-injection devices (CID cameras), which have greater radiation tolerance. Radiation concerns may require some additional shielding to be added upstream of the CCD cameras 150 in FIG. 6, to shield them against neutrons that might be produced in the beam delivery equipment. However, the elimination of patient-specific apertures 11 and compensators 12 in pencil beam scanning approaches to ion beam therapy, and the replacement of range modulators 16 in FIG. 1 by an energy selection system upstream of the beam delivery gantry in FIG. 2, promise to greatly reduce the undesired neutron dose to body parts of the patient, and therefore to the Active Water Phantom cameras 150 at their envisioned locations. The greatest potential advantage that would be offered by CIDs, then, would be resistance to possible complications arising from interactions of the ion beam within the WbLS volume that might occasionally produce neutrons at sufficiently large angles to impinge on, and “kill”, individual sensor pixels.

Embodiments of the present invention include the control computer 185 incorporating a dedicated, sophisticated software package that provides camera control and readout, data archiving and analysis, simulation of anticipated light yield profiles, and 3-D image reconstruction and visualization. The control computer 185 may be configured to simulate, based on a given ion beam therapy treatment plan, each two-dimensional light-yield profile projection as the light-yield profiles would be collected by the plurality of light-detecting devices 150. In some embodiments, the simulated light-yield profile projections correspond with those light-yield profiles measured by each of the plurality of light-detecting devices within approximately 2% of the measured light-yield profiles, when all of the hardware and software controlling the pencil beam scanning delivery are functioning correctly. More generally, the control computer 185 may be configured to provide a quantitative comparison of the measured light-yield profiles and simulated light-yield profile projections. In certain embodiments, the control computer 185 is configured to provide offline reconstruction and visualization of a three-dimensional image of the full treatment dose field that generated the three orthogonal two-dimensional light-profile views provided for each energy layer of the treatment plan by the plurality of light-detecting devices 150 and said processing electronics.

The camera control software must ensure simultaneous exposures of the three cameras 150 or photo-detector arrays viewing the Active Water Phantom from different directions. It should allow these exposures to be initiated, either singly or in time-lapsed bursts, by receipt of an external trigger signal associated with ion beam delivery. The length of exposures can be controlled either by software parameters or by the width of the external trigger signals. The raw data recording digitized charge for every pixel can be read out for individual exposures at rates of at least 10 frames per second, and recorded on a control computer disk.

The control computer 185 and associated simulation software will be capable of interfacing to pencil beam scanning treatment planning software, and of predicting, within approximately 2% accuracy, the light yields that would be measured by the readout cameras or photo-detectors when all of the ion beam delivery hardware and software is functioning correctly. These predictions will take into account all of the independently measured properties of the Active Water Phantom optical system and of the WbLS material, including the quenching of light yield when the beam ionization density within the WbLS material is high. The control computer 185 and software will allow rapid online comparisons of predicted to measured light yield profiles, to provide early signals of possible problems in the treatment plan implementation.

The control computer 185 and data analysis software will be capable of offline reconstruction and visualization of the 3-D image of the delivered dose field, exposure by exposure and summed over all the exposures in a treatment plan. It will reconstruct 3-D dose images from the three recorded orthogonal 2-D light yield profiles for each exposure, taking into account, as necessary, the known shape of the energy deposition profile with depth for an ion beam of the energy corresponding to that exposure. It will furthermore include in the reconstruction the independently measured WbLS and optical system characteristics that allow the 3-D dose distribution to be inferred from the measured 3-D light yield distribution. From comparison of the reconstructed 3-D dose image with that intended in the treatment plan, the Active Water Phantom software will generate a verification signal that indicates whether or not the treatment plan implementation meets clinical acceptance criteria to be entered by the user.

Embodiments of the present invention incorporate features that include, without limitation: (1) the capability to determine the three-dimensional radiation dose delivered by an ion beam therapy treatment plan to a tissue-equivalent medium; (2) the capability to carry out stringent tests of all aspects of a pencil beam scanning treatment implementation by providing 3-D dose images for each individual energy layer within the treatment; (3) the capability to measure the 3-D distribution with an absolute accuracy of 1-2%, and with a spatial resolution of 1-2 millimeters over the largest tumor volumes and the full range of tumor depths to be subjected to ion beam therapy, and to make these measurements for a given dose field in the same amount of time that would be required for the patient treatment, with no changes needed to the detector during the measurements; (4) the capability to provide up to four orders of magnitude more data characterizing a pencil beam scanning treatment than currently available quality assurance systems, at no greater equipment cost to the customer and in measurement times that are even shorter, to facilitate greater patient throughput; (5) the capability to span a dynamic range of approximately 1000:1 in maximum-to-minimum dose ratios within a given dose field; (6) the capability to optimize the matching of scintillation light intensity to readout sensor performance and the need to meet clinical requirements by adjustments of liquid scintillator concentration and optical system design; (7) the use of a scintillating medium that is subject to much less light scattering and attenuation, and to far less chemical, corrosiveness, and combustibility hazards than conventional liquid scintillator materials.

In broad embodiment, the present invention is an Active Water Phantom that simulates human body tissue and scintillates to reveal the three-dimensional radiation dose delivered by an intensity-modulated ion beam therapy treatment plan prior to its delivery to a patient. The scintillating medium is a newly developed water-based liquid scintillator with tissue-equivalent density and chemical composition. The 3-D information is provided with dose measurement accuracy and spatial resolution meeting clinical requirements, by readout cameras or photo-detectors viewing three orthogonal 2-D projections of the light yield generated by an ion beam stopping in the medium. The readout of the cameras or photo-detectors is sufficiently fast to allow quality assurance on each individual energy layer of a pencil beam scanning treatment plan, as well as on the overall plan. The associated software provides sophisticated control and readout of the cameras or photo-detectors, data archiving and analysis, simulation capabilities interfacing to patient treatment planning software, and 3-D image reconstruction and visualization.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An Active Water Phantom comprising:
a water-tight tank with a plurality of transparent walls and a plurality of opaque walls;
a water-based liquid scintillator completely filling said tank, wherein the water-based liquid scintillator is configured to simulate human tissue, and configured to emit scintillation light upon irradiation by an ion beam, to reveal the three-dimensional spatial dose distribution that would be delivered to a patient by the ion beam;
a plurality of light-detecting devices to provide three mutually orthogonal real-time measurements of the scintillation light intensity emitted from said water-based liquid scintillator, each measurement a function of a two-dimensional position; and
processing electronics configured to digitize the signal outputs from each of the plurality of light-detecting devices.

2. The Active Water Phantom of claim 1, wherein the water-based liquid scintillator is sized to accommodate the largest and deepest tumor locations to be treated by ion beam therapy.

3. The Active Water Phantom of claim 1, wherein the water-based liquid scintillator comprises a concentration of organic scintillating molecules attached to water molecules by means of a non-ionic surfactant bridge.

4. The Active Water Phantom of claim 3, wherein the organic scintillating molecules comprise one of linear alkylbenzene (LAB), trimethyl benzene (pseudocumene or PC), di-isopropylnaphthalene (DIN), phenylxylylethane (PXE), and phenylcyclohexane (PCH).

5. The Active Water Phantom of claim 4, wherein the water-based liquid scintillator further comprises fluorescent material and/or other wavelength-shifting materials such that light emitted by the organic scintillating molecules is shifted toward longer wavelengths to improve a match with the spectral sensitivity of the plurality of light-detecting devices.

6. The Active Water Phantom of claim 5, wherein the water-based liquid scintillator material, including any fluorescent and wavelength-shifting admixtures, has a density within 1% of the density of pure water and matches the composition percentages, by weight, of hydrogen, oxygen and carbon in normal human body tissue sufficiently well that the ion beam energy deposition in human tissue can be predicted within an accuracy of 1% from the measured ion beam energy deposition in the water-based liquid scintillator.

7. The Active Water Phantom of claim 3, wherein the non-ionic surfactant bridge is sulfonic acid.

8. The Active Water Phantom of claim 3, wherein the non-ionic surfactant is optimized, for a given choice of organic scintillating molecules, to chemically suppress radiation-induced free radical formation in the water-based liquid scintillator.

9. The Active Water Phantom of claim 8, wherein anti-oxidants may be added to the mixture to further suppress free radical formation.

10. The Active Water Phantom of claim 3, wherein the water-based liquid scintillator is configured such that less than 1% of the emitted light is attenuated by passage through the water-based liquid scintillator on its path toward each of the plurality of light-detecting devices.

11. The Active Water Phantom of claim 1, wherein the reflectance of an inner surface of each of the plurality of transparent walls and each of the plurality of opaque walls for wavelengths within the emission spectrum of said water-based liquid scintillator is less than four percent.

12. The Active Water Phantom of claim 1, wherein each of the plurality of transparent walls of said water tank transmits at least 80% of the scintillation light to its respective one of the plurality of light-detecting devices, while each of the plurality of opaque walls prevents external light from entering an interior volume of said water tank.

13. The Active Water Phantom of claim 12, wherein each of the plurality of transparent walls is configured to act as an optical lens to transport light, generated at different distances from the transparent wall, along a line perpendicular to the transparent wall, up to a depth of field as deep as 30 centimeters, to illuminate a spot on the respective remote camera sensor whose dimensions are comparable to, or smaller than, the effective sensor pixel size used.

14. The Active Water Phantom of claim 12, further comprising a plurality of optical fibers optically coupled to one or more of the plurality of transparent windows to transmit the generated scintillation light to remote photo-detectors.

15. The Active Water Phantom of claim 1, wherein scintillation light is transported from the water tank to the plurality of light-detecting devices through hoods configured to eliminate light from sources external to the water tank, the hoods having inner wall surfaces designed to minimize reflections of the generated scintillation light.

16. The Active Water Phantom of claim 1, wherein the optical transport system has sufficient resolution and each of the plurality of light-detecting devices is configured with a sufficient number of channels to provide a two-dimensional view of the dose field that distinguishes light originating from neighboring pixels separated by no more than 1-2 millimeters within the water-based liquid scintillator, when light origination points, within each pixel, span a depth of field up to 30 centimeters.

17. The Active Water Phantom of claim 16, wherein the processing electronics are configured to digitize output signals from each of the plurality of light-detecting devices in a sufficient number of bits, and with sufficiently low readout noise, to span a maximum-to-minimum dynamic range of approximately 1000:1 in a light yield generated from a given pixel.

18. The Active Water Phantom of claim 16, wherein the digitized output signal from each channel of the plurality of light-detecting devices is proportional to a light yield from a corresponding pixel within the water-based liquid scintillator, within a precision of approximately 1%, up to a maximum light yield anticipated for ion beam therapy treatment plans.

19. The Active Water Phantom of claim 16, wherein the digitized output signals from all channels are read out, by the processing electronics, and stored at least ten times per second, to provide 3-D dose profile measurements for each independent energy layer in a Pencil Beam Scanning treatment plan.

20. The Active Water Phantom of claim 16, wherein exposures of the plurality of light-detecting devices may be triggered externally by signals related to the ion beam being incident on the active water phantom, or triggered internally according to a software-selected preset sequence of exposures.

21. The Active Water Phantom of claim 1, further comprising a control computer configured to provide simultaneous exposures from the plurality of light-detecting devices, wherein exposure lengths are determined either by software input or by a width of external trigger signals, and wherein the control computer is configured to store data from the plurality of light-detecting devices at readout rates of at least 10 frames per second.

22. The Active Water Phantom of claim 1, wherein a concentration of scintillating molecules is arranged, and a light collection efficiency of the optical system and a quantum efficiency of the plurality of light-detecting devices are adjusted, in order to provide a statistical precision of approximately 1% in a measurement of maximum anticipated light yield from a pixel within the water-based liquid scintillator, in exposure times of 100 milliseconds or less.

23. The Active Water Phantom of claim 1, further comprising a control computer configured with software to simulate, based on a given ion beam therapy treatment plan, the two-dimensional light yield profile projections that would be collected by the plurality of light-detecting devices.

24. The Active Water Phantom of claim 23, wherein the simulated light yield profile projections correspond with those light yield profiles measured by each of the plurality of light-detecting devices within approximately 2% of the measured light yield profiles when all of the hardware and software to implement the treatment plan are functioning correctly.

25. The Active Water Phantom of claim 23, wherein the control computer is configured with software to provide a quantitative comparison of the measured light yield profiles and simulated light yield profile projections.

26. The Active Water Phantom of claim 23, wherein the control computer is configured with software to provide offline reconstruction and visualization of a three-dimensional image of the full treatment dose field that generated the three orthogonal two-dimensional light profile views provided for each energy layer of the treatment plan by the plurality of light-detecting devices and said processing electronics.

27. The Active Water Phantom of claim 23, wherein the control computer is configured with software to generate a verification signal, that indicates whether or not the treatment plan implementation meets clinical acceptance criteria, based on the detailed comparison of the 3-D dose profiles, for each energy layer individually and for the sum of all energy layers, reconstructed from the Active Water Phantom measurements with those anticipated in the treatment plan.

28. The Active Water Phantom of claim 23, wherein the control computer is configured to implement a complete three-dimensional Quality Assurance measurement for a clinical treatment plan by exposing the plurality of light-detecting devices to light from the water-based liquid scintillator when the ion beam irradiates the phantom in the same manner, and for the same irradiation times, as would be used for a human patient.

29. The Active Water Phantom of claim 1, further comprising a positioning device configured to adjust the location and orientation of the active water phantom in concert with the position and orientation of an ion beam delivery gantry.

30. The Active Water Phantom of claim 1, wherein the plurality of light-detecting devices comprises one of a plurality of CCD cameras, CID cameras, avalanche photodiodes, and photomultiplier tubes.
